(19) United States
(12) Patent Application Publication
(10) Pub. No.: US 2024/0407412 A1
(43) Pub. Date: Dec. 12, 2024

(54) Compositions and Uses Thereof

(71) Applicants: Charles Bavington, Halifax (GB); Thomas Brudenell-Bruce, Halifax (GB)

(72) Inventors: Charles Bavington, Halifax (GB); Thomas Brudenell-Bruce, Halifax (GB)

(21) Appl. No.: 17/604,629

(22) PCT Filed: Apr. 20, 2020
(86) PCT No.: PCT/GB20/50985

§ 371 Date: Oct. 18, 2021

(30) Foreign Application Priority Data
Apr. 18, 2019 (GB) 1905593.8

(51) Int. Cl. A23L 27/30; A21D 2/18; A21D 2/26; A23G 1/40; A23G 1/44; A23G 3/42; A23G 3/44; A23L 27/24; C12N 1/06

(52) U.S. Cl. CPC A23L 27/31; A21D 2/18; A21D 2/267; A23G 1/40; A23G 1/44; A23G 3/42; A23G 3/44; A23L 27/24; C12N 1/06

(57) ABSTRACT

The present invention relates to a composition comprising a cell wall extract or fraction obtained from protists. The invention also relates to a sugar replacer or low calorie or substantially calorie-free filler or bulking agent comprising a cell wall extract or fraction wherein the cell wall extract or fraction comprises protein, oil and carbohydrate. The invention also provides methods of producing a composition comprising a cell wall extract or fraction and a uses thereof.

### TECHNICAL FIELD OF THE INVENTION

The invention relates to compositions comprising a protist cell wall extract or fraction suitable for providing a low calorie or substantially calorie-free alternative to saccharides used in food products. The present invention also relates to a method of producing the composition comprising a protist cell wall extract or fraction.

### BACKGROUND TO THE INVENTION

Sugar, a soluble carbohydrate with a sweet taste, is used in nutritional products and can also be utilised in the pharmaceutical and cosmetic industries. A variety of sugar types can be obtained from different sources from simple monosaccharide sugars (glucose and fructose), disaccharides (sucrose) to complex longer chain oligo and poly-saccharides. Saccharide molecules generally conform to the empirical formula CₙH₂ₙOₙ. Each monomer of a complex poly-saccharide is each joined by a glyosidic bond. Carbohydrate molecules are natural polymers that are responsible for many roles within living organisms from energy storage to providing structural support. Other saccharide molecules play fundamental roles in the immune system, disease pathogenesis and development.

Refined sucrose is a common ingredient, present in many food and drinks products. Even some low fat food options contain a high sugar content to improve taste. Public Health England recommends no more than 30 g of processed sugar should be consumed each day (approximately 5% of the total daily calories). However recent reports suggest that the average amount of sugar consumed is more than double the recommended amount. Diets with high processed sugar content have been implicated in obesity, diabetes, tooth decay and cardiovascular disease. Therefore, there is a need for the inclusion of low calorie or substantially calorie-free carbohydrate alternatives in the modern diet.

A carbohydrate product that is low or calorie-free must be non-digestible, i.e. it is not hydrolysed by digestive enzymes in the small intestines. This is due to a lack of enzymes with the capability to hydrolyse the B1-4 linkage of some oligo-saccharide molecules. Some non-digestible products are also prebiotic as they undergo fermentation by commensal gastrointestinal bacteria which induce specific changes in activity within the gut. Where the oligosaccharide is resistant to both digestion and fermentation, it passes through the gut and into faeces, thus providing no nutritional or calorific value. This is of particular interest in the development of low or calorie-free food products.

Due to their widespread abundance saccharides can be obtained from multiple sources including plant and animal tissue and microbial cells such as bacteria, algae and fungi. Gum Arabic, Hyaluronic acid, Sucrose, Xanthan gum and sucrose are just some examples of sugar containing products that can be isolated from natural sources. In 2016, the combined world production of sugarcane and sugar beet, from which sucrose is isolated, was approximately two billion tonnes.

An object of the invention is to provide a composition which can be used to replace at least a proportion of sugar in food products for human and non-human mammal consumption.

A further object of the invention is to provide a composition that can be used as a low calorie or substantially calorie-free filler or bulking agent in food products for human and non-human mammal consumption.

An additional object of the invention is to provide methods and uses thereof, of compositions for use as a sugar replacer or low calorie or substantially calorie-free filler in products suitable for human and non-human consumption.

### SUMMARY OF THE INVENTION

The invention relates to compositions obtained from microbial cells.

Particularly, the invention relates to compositions obtained from protist cells.

In accordance with the first aspect of the invention, there is provided a composition comprising a protist cell wall extract or fraction.

In accordance with a second aspect of the invention, there is provided a sugar replacer or low calorie or substantially calorie-free filler composition comprising a protist cell wall extract or fraction.

Many strains of protists (including those commonly referred to as microalgae) are generally regarded as safe (GRAS) and the sustainable production methods associated with their growth makes them a favourable candidate for use in biotechnological processes.

As used herein, a “microbial cell” or “microorganism” refers to organisms such as algae, bacteria, fungi, yeast, protist, and combinations thereof, e.g., unicellular organisms. In some embodiments, a microbial cell is a eukaryotic cell. A microbial cell includes, but is not limited to, golden algae (e.g., microorganisms of the kingdom Stramenopiles); green algae; diatoms; dinoflagellates (e.g., microorganisms of the order Dinophyceae including members of the genus Crypthecodinium such as, for example, Crypthecodinium cohnii or C. cohnii); microalgae of the order Thraustochytriales; The microbial cells are from microalgae of the order Thraustochytriales, which includes, but is not limited to, the genera Thraustochytrium and/or Aurantiochytrium.

The inventors surprisingly found that it is possible to isolate a novel carbohydrate prepared from a cell wall extract or fraction of marine protists of the order Thraustochytriales, which includes the genus Aurantiochytrium. Preferably the Aurantiochytrium comprises Aurantiochytrium sp. A2 (CCAP 4062/9) or derivatives or mutant strain thereof. Such extracts or fractions have been found to be non-digestible and non-fermentable, and do not illicit an immune response. The cell wall preparation can be used to replace all or part of the sugar present in a wide range of foodstuffs, including confectionary and baked goods formulations, providing favourable texture, taste and mouthfeel, with a reduced calorie content and glycaemic index.

The invention relates to a composition comprising a cell wall extract or fraction. Preferably the cell wall extract or fraction is a non-digestible, non-prebiotic polysaccharide or oligosaccharide isolated from cell wall preparations of protist cells such as those of the order Thraustochytriida. Such a composition will find applications as a sugar replacer or low calorie or substantially calorie-free fillers for use in low or substantially calorie free food products. The composition may replace some or substantially all of the sugar compound of a product suitable for consumption, so as to reduce the calorie content of the food. A yet further object of the invention is to provide a method of producing the composition comprising a cell wall preparation from protist cells.

The term “food” or “foodstuff” is intended to mean any product which is intended for consumption.

In one embodiment, the cell wall extract or fraction may comprise protein, oil and carbohydrate. Preferably, the carbohydrate has a backbone structure of 1-2αGalf.

In one embodiment, the cell wall extract or fraction is substantially un-hydrolysable by known mammalian hydrolase enzymes. Such enzymes include, but are not limited to classes α-Amylase and Glucan, 1,4-α-glucosidase.

In yet another embodiment, the cell wall extract or fraction is substantially non-digestible. In additional embodiments, the cell wall extract or fraction is substantially non-fermentable. Preferably, the cell wall extract or fraction is both substantially non-fermentable and non-digestible.

In another embodiment the cell wall extract or fraction has a molecular weight ranging from about 40 kDa to about 2000 kDa. Preferably, the cell wall extract or fraction has a molecular weight of about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 500, about 600, about 700, about 800, about 900, about 1000, about 1100, about 1200, about 1300, about 1400, about 1500 up to about 1800 kDa. More preferably, the cell wall extract or fraction has a molecular weight of about 360 about 1800 kDa.

The cell wall extract or fraction of the present invention may comprise:
a) about 5 to about 40 wt % protein;
b) about 50 to about 90 wt % carbohydrate.

In a preferred embodiment the cell wall extract or fraction further comprises about 70 to about 90 wt %, about 60 to about 70 wt %, about 50 to about 60 wt %. Most preferably the cell wall extract or fraction may comprise about 70 to about 90 wt % carbohydrate.

In a preferred embodiment the cell wall extract or fraction may further comprise about 30 to about 40 wt %, about 20 to about 30 wt %, about 10 to about 30 wt % protein. Most preferably the cell wall extract or fraction may comprise about 10 to about 30 wt % protein.

In a further embodiment, the cell wall extract or fraction can be hydrolysed to yield one or more oligosaccharides. Preferably, the oligosaccharides comprises up to 10 monosaccharide units. Hydrolysis of the cell wall extract or fraction may be conducted by standard methods, for example, acid hydrolysis.

In a further embodiment, oligosaccharides have a molecular weight ranging from 300 to 2000 Da. Such low molecular weight oligosaccharides are advantageous in applications where high molecular weight molecules are unsuitable. For example as a sugar replacer or low calorie filler in foodstuffs.

In a further embodiment, the cell wall extract or fraction can be hydrolysed to yield one or more polysaccharides. Preferably the polysaccharides comprise more than 10 monosaccharide units. Hydrolysis of the cell wall extract or fraction may be conducted by standard methods, for example, acid hydrolysis.

In a further embodiment, polysaccharides have a molecular weight ranging from 1 to 60 kDa. Preferably, the polysaccharide has a molecular weight of about 1, about 5, about 10, about 15, about 20, about 25, about 30, about 35, about 45, about 35, about 40, about 45, about 50, about 55, about 60. More preferably, the cell wall extract or fraction has a molecular weight of about 1.8 to about 40 kDa.

In a preferred embodiment, each monosaccharide unit can be selected from, but is not restricted to, Arabinose, Rhamnose, Fucose, Xylose, Iduronic acid, Galacturonic acid, Mannose, Galactose, Glucose and Glucuronic acid.

In a further embodiment, the composition comprising a cell wall extract or fraction may extracted from a protist biomass comprising:
a) about 10-about 80 wt % protein
b) about 15-about 70 wt % oil
c) about 5-about 40 wt % carbohydrate

In a preferred embodiment the composition comprising a cell wall extract or fraction may be extracted from a protist biomass which may further comprise about 70-about 80 wt %, about 60-about 70 wt %, about 50-about 60 wt %, about 40-about 50 wt %, about 30-about 40 wt %, about 10-about 30 wt % protein. Most preferably the protist biomass may comprise about 10-about 30 wt % protein.

In a preferred embodiment the composition comprising a cell wall extract or fraction may be extracted from a protist biomass which may further comprise about 50 wt %, about 30-about 40 wt %, about 15-about 30 wt % oil. Most preferably the protist biomass may comprise about 15-about 30 wt % oil.

In a preferred embodiment the composition comprising a cell wall extract or fraction may be extracted from an protist biomass which may further comprise about 30-about 40 wt %, about 20-about 30 wt %, about 15-about 30 wt % carbohydrate. Most preferably the cell wall extract may comprise about 15-about 30 wt % carbohydrate.

In yet another embodiment, the composition comprising a cell wall extract or fraction may be used as a filler or bulking agent. Preferably, the filler or bulking agent comprises a cell wall extract or fraction wherein the cell wall extract or fraction comprises protein and carbohydrate.

In further embodiments the composition comprising a cell wall extract or fraction may be suitable for use in a product suitable for consumption by a human.

In such embodiments, a food product suitable for consumption by a human may be in solid, frozen, semi-solid, gel, molten, semi-liquid, liquid or powder form. Most preferably, the food product suitable for consumption by a human can be selected from one or more of, but is not limited to, pastry, cakes, biscuits, doughnuts, bread, cream, buttercream, ice-cream, jams, jellies, yogurt, custard, syrup, glaze, paste, sauces, coatings, cereal, pancakes, waffles, butter, nut-butter, chocolate spread and bagels.

In certain embodiments, the food product may comprise confectionery, for example chocolate. Suitable chocolate includes dark, milk, white and compound chocolate. In some embodiments, the confectionery is chewing gum, bubble gum or gum base. In other embodiments, the confectionery is candy. Suitable candy includes hard candy, chewy candy, gummy candy, jelly candy, toffee, fudge, nougat and the like.

If the food product comprises chocolate, then the chocolate may comprise at least one traditional or artificial sweetener. Such sweeteners include sugars (e.g. sucrose, dextrose, glucose syrup solids, fructose, lactose and maltose and any combination thereof), sugar alcohols (e.g. sorbitol, xylitol, erythritol, mannitol, lactitol, isomalt and maltitol, or any combination thereof), intense sweeteners (e.g. aspartame, acesulfame-K, cyclamates, saccharin, sucralose, neohesperidin, dihydrochalone, alitame, stena sweeteners, glycyrrhizin, or any combination thereof) and any combination of sugars, sugar alcohols and intense sweeteners.

The food product may comprise a reduced calorie chocolate or chocolate flavoured product.

The cell wall extract or fraction may be mixed with ingredients typically used to manufacture chocolate prior to or during tempering. The cell wall extract or fraction may be pre-mixed with a tempering and/or non-tempering fat. Examples of tempering fats include cocoa butter and cocoa butter equivalents. Typically, cocoa butter equivalents are blends of vegetable fat such as palm oil mid-fraction, illipe butter, shea stearine and cocoa butter. Sal, mango, and/or kolum fats may also be used. Examples of non-tempering fats include palm oil, palm kernel oil, butterfat, cocoa butter replacers and cocoa butter substitutes. Typically, cocoa butter replacers are hydrogenated, fractionated fat blends from soybean oil, rapeseed oil, palm oil, cottonseed oil, and/or sunflower oil, or other similar fats. Typically, cocoa butter substitutes are high lauric acid-containing fats, such as hydrogenated, fractionated fat blends from coconut and/or palm kernel oil, or other similar fats. The cell wall extract or fraction may be introduced into chocolate crumb if such a process is used in the manufacture of the chocolate product.

In an additional embodiment, the composition comprising a cell wall extract or fraction may be used in a pharmaceutical or nutraceutical formulation. This could be as a coating or additive for a tablet and/or in a liquid suspension to replace or supplement existing flavourings.

In a further embodiment, the composition may be added to the food product suitable for consumption by a human as a raw ingredient, by a manufacturer, prior to any one of baking, cooking, poaching, solidifying, melting, frying, boiling, caramelising, grilling, mixing, freezing, chilling, heating, crystallising.

In a further embodiment, the composition is added to the product suitable for consumption by a human. Preferably, this may be in the form of a sachet, jar, box, packet, canister and/or bag. The composition may be in the form of a powder, granules, stick, syrup, glaze or sauce.

In a further embodiment, the composition comprising a cell wall extract or fraction may be used as a direct replacement of at least a portion of the sugar and/or as a supplement. When used as a supplement, the composition comprising a cell wall extract or fraction may be present at a percentage ratio of between about 0 to about 100% with any existing sugar in a product.

Alternatively, the composition comprising a cell wall extract or fraction may be used in a food suitable for consumption by a non-human animal.

In a further embodiment, the cell wall extract or fraction may be obtained from protists of the order Thraustochytriida, preferably from the family Thraustochytriidae, and preferably from the genus Aurantiochytrium.

In a further aspect of the invention, there is provided a method for making a composition comprising a cell wall extract or fraction obtained from protist cells, the method comprising;
a) growing protist biomass by fermentation;
b) enzymatically hydrolysing the biomass to obtain a hydrolysate;
c) separating the oil phase, water phase and solid phase from said hydrolysate;
d) concentrating the water phase so as to obtain a cell wall extract or fraction.

Preferably, step b is carried out by a protease enzyme. The enzyme may be one of serine, cysteine, threonine, aspartic, glutamic proteases, metalloproteases or asparagine peptide lyases. More preferably the enzyme can be selected from any one of papain, trypsin, pepsin or alcalase.

In another embodiment, the protease enzyme is present at a concentration of about 0.05 to about 1.5% (v/v).

According to the present embodiment, the hydrolysate of step b) is heated to 80-90 degrees Celsius and centrifuged.

In an additional embodiment, the water phase is passed through filter paper or equivalent filter material to obtain a filtered water phase.

In a further embodiment, the filtered water phase is purified by tangential flow filtration using a membrane with a molecular weight cut-off of 100 kDa or less.

In an additional embodiment, the purified water phase is concentrated by tangential flow filtration by 2 fold, 3 fold, 4 fold, most preferably up to 5 fold and dried. Optionally, the concentrated sample may be precipitated in methanol, ethanol or acetone or a mixture thereof.

According to the present invention, there is also provided a method of making products suitable for human consumption comprising adding a cell wall extract or fraction obtained from protist cells such as those of the genus Aurantiochytrium as an alternative to sugar or to replace some or substantially all of the sugar.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Various embodiments of the present invention will now be described in the following, non-limited examples and with reference to the accompanying figures.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. It will be apparent to the skilled person that a number of the features of the compositions and methods listed in respect of the various aspects of the invention are interchangeable.

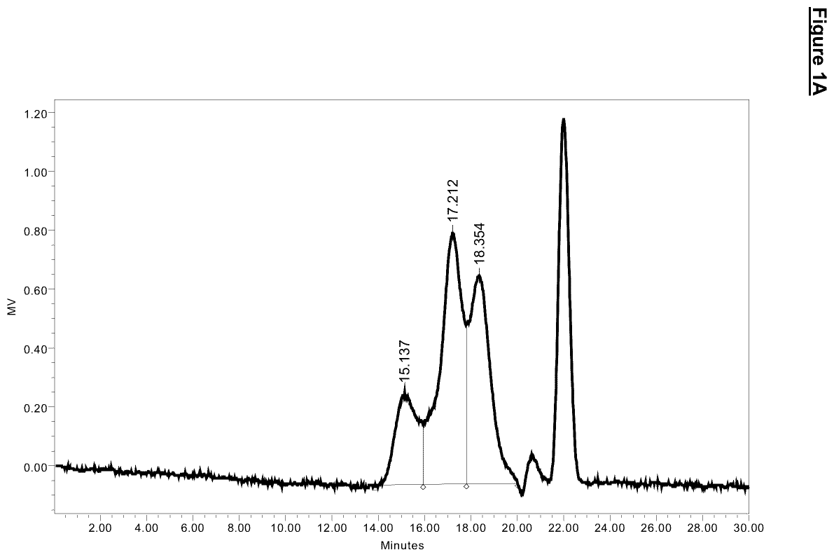

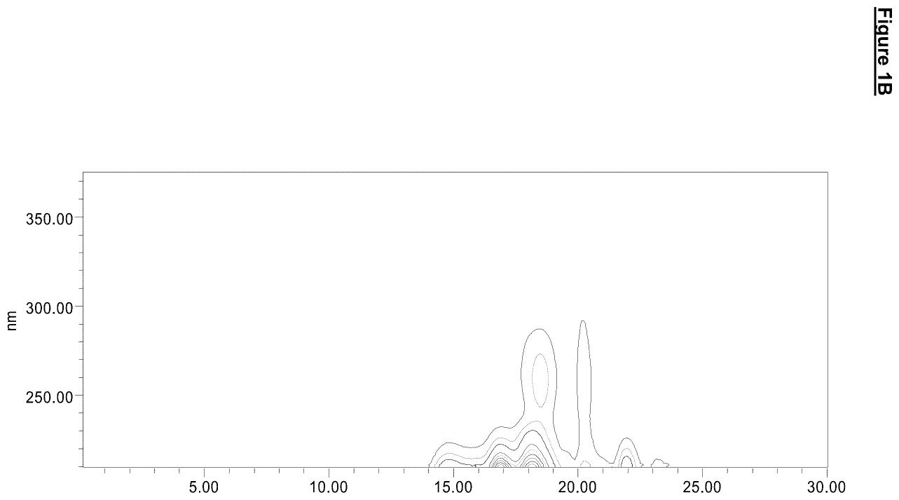

FIG. 1: A) HPLC-SEC chromatography of cell wall extract or fraction: refractive index chromatogram and B) UV absorbance on the 200-300 nm range;

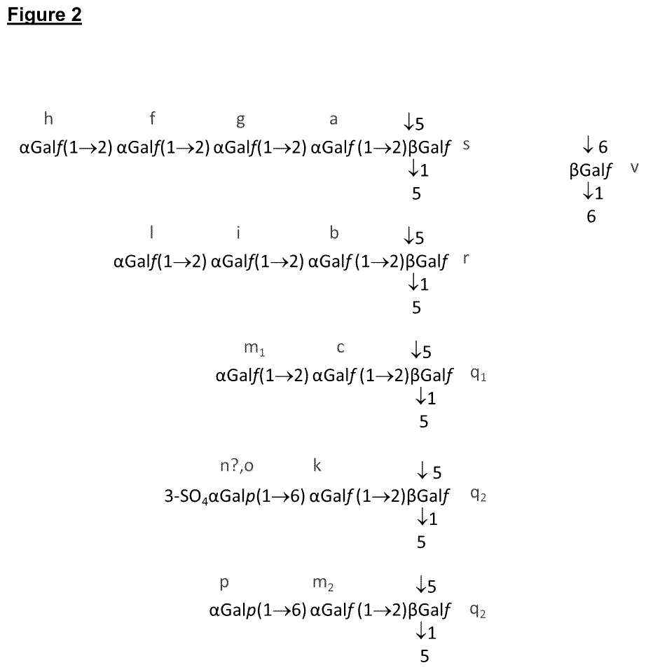

FIG. 2: Summary of the identified structures;

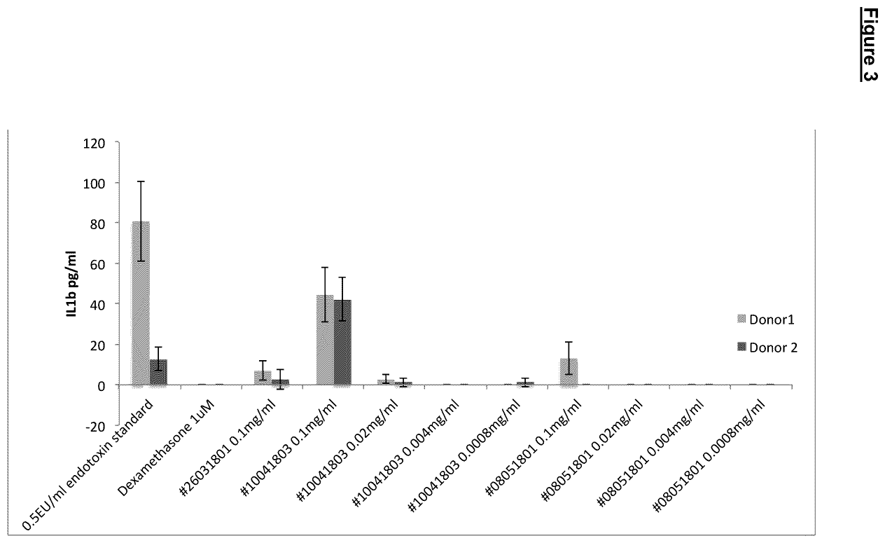

FIG. 3: IL-1b release from human whole blood treated with controls and cell wall extract or fraction; and

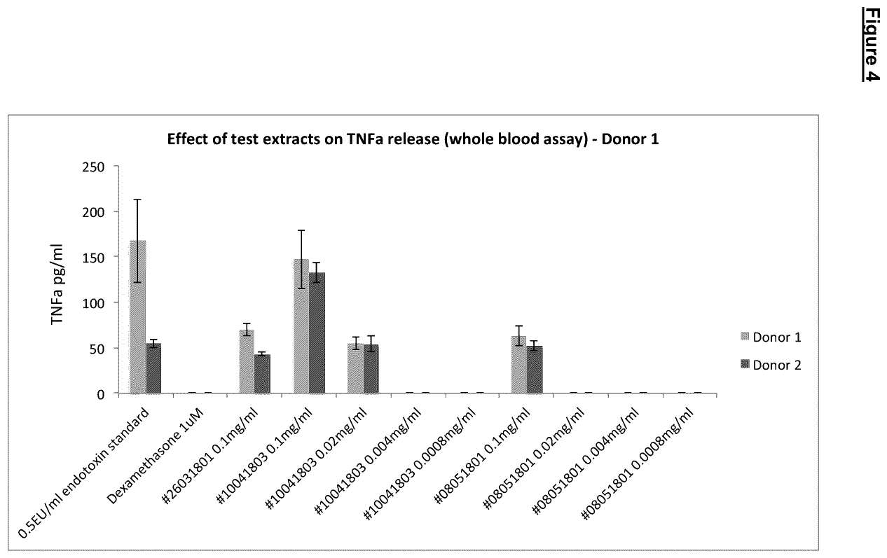

FIG. 4: The effect of carbohydrate extracts on TNFα release from human blood.

### EXAMPLES

### Example 1

### Cell Wall Extract or Fraction and Purification

The biomass from Aurantiochytrium sp. A2 (CCAP 4062/9) can be processed from dry or wet starting material. If dry biomass is used water may be added to achieve about 60-70% moisture content. The biomass may then be hydrolysed by treatment with 0.1-1.0% (v/v) Alcalase 2.4 L at 60° C. for 3-16 hours. The hydrolysate may then be heated to 80-90° C. and centrifuged. The aqueous layer may be collected and passed through filter paper. The aqueous fraction may then be purified by tangential flow filtration using a membrane with a pore size of 100 kDa or less. The purified concentrate (2-3 fold) may then be dried directly. Optionally the purified sample can be precipitated by the addition of 1 volume of methanol, ethanol or acetone, or even a mixture of such solvents. The precipitated material may be further dried to eliminate all residual solvent. The dried cell wall extract or fraction may be milled to achieve a suitable particle size to assist in formulation.

### Composition

The dried cell-wall extract may have the following composition:

TABLE 1
Proximate analysis of cell wall extract or fraction.

| | Carbohydrate | Protein | Oil |
|---|---|---|---|
| % composition | 58% | 38% | 1% |
| (dry matter | | | |
| basis) | | | |

The cell wall extract or fraction was found to have a molecular weight ranging from 40 kDa to >1400 kDa, with 2 dominant species with molecular weights of around 250 kDa and around 45 kDa corresponding to the carbohydrate and protein components as is shown in FIG. 1.

The carbohydrate may have the following monosaccharide composition listed in table 2 below.

TABLE 2
Monosaccharide composition analysis

| | 10041803 | 08051801 |
|---|---|---|
| | (>100 kDa | (>100 kDa |
| Biochemical assay | TFF) | TFF) |
| Monosaccharide %: | | |
| Arabinose | 0 | 0 |
| Rhamnose | 0.6 | 0 |
| Fucose | 0 | 0 |
| Xylose | 7.8 | 11 |
| Iduronic acid | 0 | 0 |
| Galacturonic acid | 0 | 0 |
| Mannose | 14.3 | 15 |
| Galactose | 74.5 | 71.5 |
| Glucose | 0 | 0 |
| Glucuronic acid | 1.4 | 1.8 |
| N-acetyl galactosamine | 0 | 0 |
| N-acetyl glucosamine | 0.4 | 0.8 |
| % recovery | 23.9 | 24.7 |

### Carbohydrate Structure

NMR analysis identified the dominant carbohydrate backbone as 1-2 linked αGalf as shown in FIG. 2.

### Functional Properties

As no known carbohydrate enzymes which degrade this backbone have been identified, it is therefore believed that the carbohydrate is un-digestible.

The prebiotic potential of the cell wall extract or fraction was evaluated in order to determine whether it is fermented by gut bacteria. The cell wall extract or fraction did not induce any significant modification of bifidobacterial population in a human faecal fermentation assay. Therefore it is believed that this carbohydrate does not behave like a prebiotic.

TABLE 3
Quantification of bifidobaceria DNA in faecal fermentation

| | QUBIT | | average number | Fd |
|---|---|---|---|---|
| | (ng/ul) | weight | copies in 4 ng | (2 ng/ul) |
| T0 | 7 | 200 mg | 2.06E+03 | 3.5 |
| T48 Glucose | 20 | 200 mg | 4.05E+04 | 10 |
| T48 negative control | 2 | 200 mg | 5.98E+03 | — |
| T48 OMT | 2 | 200 mg | 6.88E+03 | — |
| | | | | Growth |
| | F:Dilution | N | | in log10 |
| | pre-extract | copies/gr | Log | CFU |
| T0 | 10 | 3.61E+05 | 5.56 | |
| T48 Glucose | 10 | 2.03E+07 | 7.31 | 1.75 |
| T48 negative control | 10 | 2.99E+05 | 5.48 | −0.08 |
| T48 OMT | 10 | 3.44E+05 | 5.54 | −0.02 |

The potential immunological activity of the cell wall extract or fraction was tested on whole human blood. The cell wall extract or fractions showed only very low, or no, pro-immune activity when IL-1b released from human whole blood was treated with controls and cell wall extract or fraction, as shown in FIG. 3. The cell wall extract or fraction was formulated in chocolate at an inclusion rate of 40%. The test subjects reported no adverse effects or discomfort. Therefore, it is believed that the cell wall extract or fractions have only very low, or no, pro-immune activity.

### Formulation

A protist cell wall extract can be included in a food product, for example, chocolate, as a sugar replacer or filler. Methods of making the chocolate or caramel etc. (without cell wall extracts) are well known to the skilled person and can also be found in textbooks such as Chocolate, Cocoa and Confectionery, Bernard W. Minifie third edition, which is incorporated herein by reference.

In the following formulation, 75% of the sucrose in chocolate has been replaced with a protist cell wall extract:

Milk chocolate—11% sucrose, 33% cell wall extract, 21.6% skimmed milk powder, 4% anhydrous milk fat, 22% cocoa butter, 12% cocoa liquor, 0.4% emulsifier and flavourings, 0.02% high potency sweetener sucralose.

White chocolate—11.2% sucrose, 33.8% cell wall extract, 23.6% skimmed milk powder, 4% anhydrous milk fat, 27% cocoa butter, 0% cocoa liquor, 0.4% emulsifier and flavourings, 0.02% high potency sweetener sucralose.

Dark chocolate—7.5% sucrose, 22.5% cell wall extract, 0% skimmed milk powder, 3% anhydrous milk fat, 20% cocoa butter, 46.6% cocoa liquor, 0.4% emulsifier and flavourings, 0.02% high potency sweetener sucralose.

In the following formulations, 100% of the sucrose in chocolate has been replaced with protist cell wall extract:

Milk chocolate—44% cell wall extract, 21.57% skimmed milk powder, 4% anhydrous milk fat, 22% cocoa butter, 14% cocoa liquor, 0.4% emulsifier and flavourings, 0.03% high potency sweetener sucralose.

White chocolate—45% cell wall extract, 23.57% skimmed milk powder, 4% anhydrous milk fat, 27% cocoa butter, 0% cocoa liquor, 0.4% emulsifier and flavourings, 0.02% high potency sweetener sucralose.

Dark chocolate—30% cell wall extract, 0% skimmed milk powder, 3% anhydrous milk fat, 20% cocoa butter, 46.47% cocoa liquor, 0.4% emulsifier and flavourings, 0.02% high potency sweetener sucralose.

A cell wall extract may also be used to replace some of the sugar in the preparation of other products:

Caramel—35% glucose syrup, 10.6% cell wall extract, 7% sucrose, 35% sweetened condensed milk, 12% fat, 0.4% salt.

Shortbread—125 g butter, 112 g cell wall extract, 2 g vanilla aroma liquid, 1 g salt, 1 egg yolk (20 g), 1 egg (50 g), 250 g flour, 2 g baking Powder (soda).

Cake—250 g Butter, 200 g cell wall extract, 250 g Eggs, 220 g Flour, 15 g Corn starch, 8 g Baking powder, 4 g Vanilla aroma liquid, 2 g Salt.

A number of milk chocolate products utilising the protist cell wall extract or fraction may be formulated so as to replace a portion or the majority of the sugar content. The formulations are as follows:

Formula A - Chocolate with reduced sugar content

| | | Sugar | Non-fat |
|---|---|---|---|
| Ingredient | wt % | wt % | cocoa wt % |
| Sugar (sucrose) | 40.0 | 40.0 | |
| Cocoa butter | 16.0 | | |
| Chocolate liquor | 14.0 | | 6.6 |
| Whole milk powder | 16.0 | 5.6 | |
| Milt fat, emulsifier, flavour, lactose | 4.0 | | |
| Protist cell wall extract | 10.0 | | |

Formula B - Chocolate with reduced sugar content

| | | Sugar | Non-fat |
|---|---|---|---|
| Ingredient | wt % | wt % | cocoa wt % |
| Sugar (sucrose) | 30.0 | 30.0 | |
| Cocoa butter | 16.0 | | |
| Chocolate liquor | 14.0 | | 6.6 |
| Whole milk powder | 16.0 | 5.6 | |
| Milt fat, emulsifier, flavour, lactose | 4.0 | | |
| Protist cell wall extract | 20.0 | | |

Formula C - Chocolate with reduced sugar content

| | | Sugar | Non-fat |
|---|---|---|---|
| Ingredient | wt % | wt % | cocoa wt % |
| Sugar (sucrose) | 20.0 | 20.0 | |
| Cocoa butter | 16.0 | | |
| Chocolate liquor | 14.0 | | 6.6 |
| Whole milk powder | 16.0 | 5.6 | |
| Milt fat, emulsifier, flavour, lactose | 4.0 | | |
| Protist cell wall extract | 30.0 | | |

Formula D - Chocolate with reduced sugar content

| | | Sugar | Non-fat |
|---|---|---|---|
| Ingredient | wt % | wt % | cocoa wt % |
| Sugar (sucrose) | 10.0 | 10.0 | |
| Cocoa butter | 16.0 | | |
| Chocolate liquor | 14.0 | | 6.6 |
| Whole milk powder | 16.0 | 5.6 | |
| Milt fat, emulsifier, flavour, lactose | 4.0 | | |
| Protist cell wall extract | 40.0 | | |

Formula E - Sugar-free chocolate

| | | Sugar | Non-fat |
|---|---|---|---|
| Ingredient | wt % | wt % | cocoa wt % |
| Sugar (sucrose) | 0.00 | 0.00 | |
| Cocoa butter | 20.0 | | |
| Chocolate liquor | 20.0 | | 6.6 |
| Skimmed milk powder | 16.0 | Trace | |
| Milt fat, emulsifier, flavour, lactose | 4.0 | | |
| Protist cell wall extract | 40.0 | | |

### Example 2

### 1) Initial Extraction Experiments on Spray-Dried Biomass OT-BC

The initial aim was to isolate carbohydrates from thraustochytrid biomass in sufficient yield and purity for downstream bio-activity analysis. A new extraction process was developed with spray-dried biomass (alcalase digestion, heating, centrifugation, cooling, separation of the aqueous and fat layers). Experiments were designed to recover maximum carbohydrate, without the use of solvents, which appear to interfere with carbohydrate solubility, and particularly to consider if the emulsified carbohydrate and polar lipids could be separated.

After protease treatment, heating and centrifugation the aqueous soluble material was removed for subsequent analysis. The resulting analysis of the aqueous layer indicated that a large amount of ash and protein was present (as shown in Table 4 below), and the yield of saccharide was low (˜0.5% w/w of starting material). (Proximate analysis of starting material by University of Stirling indicated ˜16% carbohydrate-Table 4). Therefore, it is believed that the carbohydrate may not have been precipitated or may have been emulsified in other layers of the sample. Proximate analysis on the creamy (polar lipid emulsion) layer several weeks later, supported that some carbohydrate was also contained in this layer (Table 4 below).

TABLE 4
Proximate analysis of fractions derived
from 1 kg spray dried biomass

| | | Prep 1 Dec17 | Prep1 | Prep1 |
|---|---|---|---|---|
| | OT-BC | Aqueous | polar lipid | neutral lipid |
| | spray dried | layer¹ | layer* | layer* |
| | g/100 g | g/100 g | g/100 g | g/100 g |
| Lab | (Stirling) | (CLS) | (CLS) | (CLS) |
| Moisture | 9.63 | 5.91 | 44 | 36.6 |
| Ash | 19.23 | 64 | 7.2 | 6.2 |
| Lipid | 25.25 | 0.18 | 19.7 | 39.8 |
| Carbohydrate | 16.22 | 15.47 | 12.4 | 4.6 |
| Protein | 36.54 | 14.43 | 16.7 | 12.9 |

¹3.9% total yield w/w from starting
*stored at room temp for 4 weeks prior to testing/yield not available

### 2) Further Evaluation of the Extraction Approach

Changes to the extraction (based on part (1)) may be required to improve carbohydrate recovery from the spray-dried biomass.

Certain carbohydrates may not precipitate out of solution with alcohol either due to the structure of the polysaccharide or the volume of alcohol used for precipitation. By avoiding the alcohol precipitation step in the isolation process there may be an increase in carbohydrate recovery. Therefore instead of precipitating the aqueous fraction it was dialysed to remove salts and other lower MW material.

To prevent emulsification of carbohydrate and polar lipids, and make subsequent carbohydrate recovery more efficient, attempts were made to remove polar lipids by solvent extraction before addition of water (aqueous conditions are required for alcalase digestion)—two samples of material were extracted separately with either ethanol or acetone/methanol (2:1 v/v) as a first step.

Emulsions are generally quite stable but can sometimes be destabilized by solvent extraction with polar solvents. In an attempt to disrupt the creamy layer emulsion, formed after protease digestion, the method described by Maximiano et al 2008 was used (acetone/methanol extraction), with some adjustments, to release carbohydrates from the creamy layer.

The yield of the non-purified aqueous fraction was 6% w/w, which was an improvement on the 3.9% achieved using a method including an alcohol precipitation step (Table 4 above). An alternative extraction protocol may be used to generate similar carbohydrate material, but purity and yields may not show an improvement compared to the aqueous soluble fraction (i.e. similar yields, but still close to 50% protein suggesting these methods would not enhance the carbohydrate recovery process). As the handling of the aqueous soluble material was more straightforward, and amenable to scale up, further work may be focused on improving the yield and purity of this fraction with respect to improving purity.

### 3) Further Method Development to Reduce Protein Content

The aqueous soluble fraction may contain a large percentage of total carbohydrate, but protein content could also be high. To further purify the carbohydrate fraction, two approaches could be taken.

The initial biomass may be washed prior to protease digestion to remove as much residual media as possible, which has been identified as a likely source of the high protein contamination. In a second experiment the aqueous fraction may be processed directly using a 100 kDa MW cut off membrane to retain carbohydrate material and remove as much protein as possible in the permeate, along with salts and other residual water soluble media components.

The amount of protein present in the fraction remained high, even after washing of the original biomass (Table 5—samples 26031802/03). Processing the aqueous fraction using tangential flow filtration (TFF) with a 100 kDa MW cut off membrane may result in reduction of protein content close to background levels (Table 5 sample 10041803). It could be seen from analysis of the 5 kDa retentate that most of the protein had been isolated in this fraction (Table 5—17041801). It was therefore clear that the introduction of a TFF step directly on the aqueous fraction may be a more effective way of further purifying the sample, and that this could be more amenable to scale up approaches.

TABLE 5
Protein content of carbohydrate fractions by BCA assay.

| | | % Protein | | |
|---|---|---|---|---|
| GlycoMar | | (by BCA | | |
| Batch No. | Description | assay) | Comment | |
| 26031801 | 12 Mar. 2018 Aqueous | 34.1 | (3.5 kDa | |
| | layer-no washing of biomass | | dialysed) | |
| 26031802 | 12 Mar. 2018 Aqueous layer - | 36.7 | (3.5 kDa | |
| | after water wash of biomass | | dialysed) | |
| 26031803 | 12 Mar. 2018 Aqueous layer - | 34 | (3.5 kDa | |
| | after salt wash of biomass | | dialysed) | |
| 10041803 | Aqueous layer (26031801) | 4.8 | 100 | kDa TFF |
| | >100 kDa TFF 27 Mar. 2018 | | | |
| 17041801 | Aqueous layer (26031801) | 25.7 | 5-100 | kDa |

5-100 kDa TFF 10 Apr. 2018

### 4) Confirmation of Method and Scale Up to 250 g Batches.

Previous method development had been carried out on 25 g batches, to allow ease of handling and preserve stocks of biomass. In order to generate sufficient material for downstream analysis, a larger scale preparation was required.

A non-solvent based protocol as determined by lab scale studies, including a TFF step with 100 kDa MW cut off membrane instead of alcohol precipitation—250 g and 623 g batches, may be used. A freeze thaw step between aqueous layer recovery and TFF processing may be introduced due to timing of the TFF runs, which take 1-2 days.

The TFF cleaned up extracts generated carbohydrate rich samples, with lower protein content than earlier preparations as seen in table 6 below. Overall the yield was still only 2-3% w/w of starting material, although this was an improvement on the 0.5% originally obtained. The MW profile (by HPLC size exclusion chromatography with RI detection using Shodex SB806M column and dextran standards) and monosaccharide composition (by methanolysis, TMS derivatisation and GC-FID) of all the batches was very similar (Tables 6 and 7 below) indicating that the extraction was consistent and reproducible.

TABLE 6
Recovery, protein content and size exclusion chromatography data for recent samples.

| GlycoMar | | | Yield % w/w | Protein % | Average MW/kDa | |
|---|---|---|---|---|---|---|
| batch # | Description | Recovery/g | (of starting) | (by BCA assay) | (dextran standards) | % peak area |
| 26031801 | 12 Mar. 2018 Aqueous layer | 1531.9 | 6.1 | 34.1 | >1400, 175.4, | 6.6, 11.1, |
| | 25 g | | | | 32.2, 2.9 | 24.6, 57.8 |
| 10041803 | Aqueous layer (26031801) >100 | 258.8 | 1.6 | 4.8 | >1400, 130.8, | 19.8, 43.0, |
| | kDa TFF 27 Mar. 2018 1 g | | | | 20.5, <1 | 36.0, 1.2 |
| 08051801 | Aqueous layer prep | 4865 | 1.9 | 10.3 | >1400, 229.9, | 19.3, 52.6, |
| | 23 Apr. 2018 >100 kDa TFF 250 g | | | | 43.4 | 28.2 |
| 16071801 | Aqueous layer prep | 18626.4 | 3.0 | tbc | >1400, 280.2, | 17.1, 50.4, |
| | 3 Jul. 2018 >100 kDa TFF 624 g | | | | 45 | 32.6 |

The resulting saccharide enriched fraction was largely soluble at lab-scale (10041803), but an insoluble opaque pellet formed with products from larger scale runs (at 1% in water). This could be due to the freeze-thaw step, which may result in poor re-solubilisation of the high molecular weight saccharide, or simply due to the extraction process more efficiently isolating high MW material.

The material appeared similar to a carbohydrate isolated in the earlier project, although there was more mannose present as show in Table 7 below. This could be associated with the more complete extraction process (spray-dried versus wet biomass starting material), which results in the isolation of cell wall manno-proteins. It can be seen that introduction of TFF reduces the mannose (which also correlates with a reduction in protein content).

In the first experiment the overall results indicated a consistent saccharide-containing product had been obtained, with two larger peaks (average ˜550-800 kDa, ˜28-39 kDa) and some smaller material (3.1, <1 kDa). In the second experiment, there appeared to be slightly more high MW carbohydrate present (17-19%>1400 kDa), an intermediate product (˜100-300 kDa) and a similar 20-45 kDa product. These differences are likely due to the more complete extraction process, and improve solubility of the product, possibly enhanced by the lack of solvent use.

TABLE 7
Monosaccharide analysis of carbohydrate extracts, compare to data from earlier studies.

| | 19 Jan. 2017 | 25 Jan. 2017 | | 10041803 | 08051801 |
|---|---|---|---|---|---|
| | IPA extract | hex extract | 26031801 | (>100 | (>100 |
| Biochemical assay | 3.5 kDa | 3.5 kDa | (3.5 kDa) | kDa TFF) | kDa TFF) |
| Monosaccharide %: | | | | | |
| Arabinose | 0.8 | 1.2 | 0 | 0 | 0 |
| Rhamnose | 0.0 | 0.0 | 1.5 | 0.6 | 0 |
| Fucose | 0.0 | 0.0 | 0 | 0 | 0 |
| Xylose | 5.0 | 5.7 | 5 | 7.8 | 11 |
| Iduronic acid | 0.0 | 0.0 | 0 | 0 | 0 |
| Galacturonic acid | 0.0 | 0.0 | 0 | 0 | 0 |
| Mannose | 6.9 | 7.6 | 26.8 | 14.3 | 15 |
| Galactose | 85.9 | 82.4 | 59.6 | 74.5 | 71.5 |
| Glucose | 1.5 | 2.1 | 5.6 | 0 | 0 |
| Glucuronic acid | 0.0 | 0.7 | 0 | 1.4 | 1.8 |
| N-acetyl galactosamine | 0.0 | 0.0 | 0 | 0 | 0 |
| N-acetyl glucosamine | 0.0 | 0.4 | 1.3 | 0.4 | 0.8 |
| % recovery | 48.4 | 33.4 | 27.2 | 23.9 | 24.7 |
| % sulphate | 8.6 | 13.1 | <3.8 | <3.8 | <3.8 |
| % protein | 5.7 | 6.3 | 34.1 | 4.8 | 10.3 |

### 5) Testing Immunological Activity of Carbohydrate Rich Extracts on Human Blood Cells.

To start an initial assessment of the potential immunological activity of the thraustochytrid carbohydrate rich samples, they were tested for their pro-inflammatory effects on whole human blood.

The whole blood assay involved incubating test samples (at 4 concentrations) with freshly isolated human blood (20 hours), and analysis of the release of the pro-inflammatory cytokines TNFα by ELISA. An endotoxin control (which is pro-inflammatory) and a dexamethasone control (which is anti-inflammatory) were included to monitor assay performance. Samples were also tested in the presence of the endotoxin control to ensure they did not interfere with the sensitivity of the assay. Two assays were carried out to allow for varying responses from blood donors (as shown in FIG. 4).

The extracts generated a weak, or no, pro-inflammatory response, in terms of the TNFα released. In the case of donor 1, the largest response in one of the samples (10041803 at it's highest loading concentration) was still below the 0.5 EU/ml endotoxin standard (the level accepted for most medical devices and pharmaceutical applications)—light grey bars as shown in FIG. 4.

In donor two only one sample generated a response above the 0.5 EU/ml standard, although the overall response by this donor was low (sample 10041803—dark grey bars as shown in FIG. 4). The differences between the different thraustochytrid extracts may have been due to their purity, or comparative solubility—i.e. sample 08051801 was not completely soluble at 1% in water, and this would result in lower loading into the blood assay.

The forgoing embodiments are not intended to limit the scope of the protection afforded by the claims, but rather to describe examples of how the invention may be put into practice.

### Biological Deposits

A deposition of biological material was made to Culture Collection of Algae and Protozoa (CCAP) for the purposes of filing one or more patent applications. Culture Collection of Algae and Protozoa (CCAP) is a recognised International Depository Authority (IDA) under the Budapest Treaty and the deposition of biological material was made on the same terms as those laid down in the Treaty. The deposit has been assigned a number along with the prefix “CCAP”.

The application refers to the following indications of deposited biological materials:
Name: Culture Collection of Algae and Protozoa
Address: SAMS Limited
   Scottish Marine Institute
   Dunbeg
   Oban
   Argyll
   PA37 1QA
   United Kingdom
Date: 15 Apr. 2019
Accession Number: CCAP 4062/9
Descriptor: Aurantiochytrium sp. A2
Depositor: Charles Bavington

1. A composition comprising a protist cell wall extract or fraction, wherein the cell wall extract or fraction comprises:
a) 5 to 40 wt % protein; and
b) 50 to 90 wt % carbohydrate.

2. The composition according to claim 1, wherein the cell wall extract or fraction comprises protein, and carbohydrate, wherein the carbohydrate has a backbone structure of 1-2αGalf.

3. The composition according to claim 2 which is substantially un-hydrolysable by mammalian hydrolase enzymes.

4. The composition according to claim 1, wherein the cell wall extract or fraction is substantially non-digestible and/or non-fermentable.

5. The composition according to claim 1, wherein the cell wall extract or fraction has a molecular weight ranging from:
a) about 40 kDa to about 2000 kDa;
b) about 30 kDa to about 1800 kDa; or
c) about 1.8 kDa to about 40 kDa.

6-8. (canceled)

9. The composition according to claim 1, wherein the cell wall extract or fraction comprises about 10 to about 30 wt % protein.

10. The composition according to claim 1, wherein the cell wall extract or fraction comprises about 70 to about 90 wt % carbohydrate.

11. The composition according to claim 1, wherein the cell wall extract or fraction can be hydrolysed to yield one or more oligosaccharides and wherein the one or more oligosaccharides comprise up to 10 monosaccharide units, selected from one or more of Arabinose, Rhamnose, Fucose, Xylose, Iduronic acid, Galacturonic acid, Mannose, Galactose, Glucose and Glucuronic acid.

12. (canceled)

13. The composition according to claim 11, wherein the oligosaccharides have a molecular weight ranging from 360 to 2000 Da.

14. The composition according to claim 1, wherein the cell wall extract or fraction can be hydrolysed to yield one or more polysaccharides, wherein the polysaccharides have a molecular weight ranging from 1 to 40 kDa.

15-20. (canceled)

21. The composition according to claim 1, for use in, or with, a food product suitable for consumption by a human or animal.

22. The composition according to claim 21, wherein the food product is in a solid, frozen, semi-solid, gel, molten, semi-liquid, liquid or powder form.

23. The composition according to claim 21, wherein the food product is selected from one or more of pastry, cakes, chocolate, candied confectionary, sherbet, caramel, toffee jelly sweets, biscuits, doughnuts, bread, cream, buttercream, ice-cream, jams, jellies, yogurt, custard, syrup, glaze, paste, sauces, coatings, cereal, pancakes, waffles, butter, nut-butter, chocolate spread and/or bagels.

24-30. (canceled)

31. The composition according to claim 21, wherein the composition is used as a supplement at a percentage ratio of between about 0 to about 100% with any existing sugar in the food product.

32. (canceled)

33. The composition according to claim 1, wherein the cell wall extract or fraction is obtained from protist is of the order Thraustochytriida.

34. The composition according to claim 33, wherein the protist is of the family Thraustochytriidae.

35. The composition according to claim 34, wherein the protist is of the genus Aurantiochytrium.

36. The composition according to claim 35, wherein the Aurantiochytrium comprises Aurantiochytrium sp. A2 (CCAP 4062/9).

37. The method for making a composition comprising a protist cell wall extract or fraction, wherein the cell wall extract or fraction comprises:
(i) 5 to 40 wt % protein; and
(ii) 50 to 90 wt % carbohydrate,
wherein the method comprises:
a) growing protist biomass by fermentation;
b) enzymatically hydrolysing the biomass with an enzyme to obtain a hydrolysate, wherein the enzyme is a protease enzyme selected from any one of papain, trypsin, pepsin, or alcalase;
c) separating the oil phase, water phase and solid phase from said hydrolysate; and
d) concentrating the water phase so as to obtain a cell wall extract or fraction.

38-46. (canceled)

47. The method according to claim 37, wherein the cell wall extract or fraction is extracted from a protist biomass comprising 15 to 70 wt % oil.
